(19) United States
(12) Patent Application Publication
(10) Pub. No.: US 2026/0123829 A1
(43) Pub. Date: May 7, 2026

(54) VISUAL EAR PICK WITH BUILT-IN WIFI, LIGHTING SUPPLEMENTATION, AND CAMERA FUNCTIONS

(71) Applicant: Jian HU, Guang'an (CN)

(72) Inventor: Jian HU, Guang'an (CN)

(21) Appl. No.: 18/938,305

(22) Filed: Nov. 6, 2024

(51) Int. Cl. A61B 1/227; A61B 1/00; A61B 1/06

(52) U.S. Cl. CPC A61B 1/227; A61B 1/000094; A61B 1/0004; A61B 1/00045; A61B 1/0684; A61B 2560/02

(57) ABSTRACT

A visual ear pick with built-in WiFi, lighting supplementation, and camera functions relates to a visual ear pick with a camera and a matching smart phone application and solves the problems that people cannot observe conditions in the ear canals when cleaning the ear canals with an ear pick. The visual ear pick is connected to a mobile phone or a tablet. The ear pick is turned on and is slightly put into the ear canal. Real-time images on the mobile phone or the tablet are observed, so that a position and angle of the ear pick are adjusted according to a need to clean the ear canals or observe conditions in the ears, thereby intuitively observing the conditions in the ears and avoiding a risk caused by a blind operation.

### TECHNICAL FIELD

The present disclosure relates to a visual ear pick with a camera and a matching smart phone application, thereby achieving a method for wirelessly transmitting an image of a camera at a head portion of the ear pick to a mobile phone and achieving reminding of being near the eardrum and wireless firmware upgrade.

### BACKGROUND

Traditionally, people clean their ear canals with ear picks, but they cannot observe conditions inside the ear canals themselves. The present disclosure achieves a personal hygiene device with a camera, such as a visual ear pick, which needs to be connected to a smart phone application of a user through a WiFi hotspot to transmit image data inside the ear canals. The car pick is provided with a built-in high-definition camera that transmits images inside the ear canal in real time to a mobile phone or a tablet through a wireless technology. The user can intuitively observe conditions inside the cars, thereby avoiding the risk caused by blind operation. By the arrangement of the high-definition camera, it ensures clear images and facilitates users to observe details inside the ear canals. Bluetooth or WiFi connection is supported. The operation is simple, and complex cable connection is not required. The ear pick is made of a food-grade material, which is easily cleaned and disinfected, ensuring hygiene and safety during use. In addition to cleaning the ear canals, the ear pick can also be used as an endoscope to observe other small internal structures.

### SUMMARY

For the shortcomings of the above technology, the present disclosure discloses a visual ear pick with built-in WiFi, lighting supplementation, and camera functions, which can intuitively transmit a video of a camera on the visual ear pick in real time to a smart phone application. The present disclosure achieves displaying of images of an ear canal on a smart device such as a mobile phone. When a user uses the visual ear pick device, the application identifies the eardrum in a timely manner by recognizing an image inside the ear canal and reminds the user to get away from the eardrum. The application can obtain new firmware through a server and has the capability of performing wireless upgrade after it is connected to a personal protective device. When the user uses a personal hygiene device with a camera, the user can establish a connection with the application more quickly and be reminded in a timely manner of a safe distance from the eardrum. Wireless firmware upgrade can be performed at any time to keep the functions being updated.

A visual ear pick with built-in WiFi, lighting supplementation, and camera functions includes the following steps:
step I, components of the device include a camera module, a lighting supplementation system, a WiFi module, a processor, a storage unit, and a user interface;
step II, application functions include image recognition, real-time displaying, and reminding;
step III, wireless upgrade functions include firmware update and wireless transmission; and
step IV, a user quickly establishes connection to an application through a QR code or by entering a device ID, and the user turns on/turns off a camera to adjust brightness by performing an operation of controlling a device through the application.

In a further technical solution of the present disclosure, a high-resolution camera is arranged in the camera module to capture an image inside the ear canal; the lighting supplementation system includes a light-emitting diode (LED) lamp or another light source to provide sufficient lighting in a low-light environment; the WiFi module is configured to achieve wireless connection between the device and a smart phone or another smart device; the processor is configured to: process image data captured by the camera and control other functions of the device; the storage unit is configured to store firmware and user data; and the user interface includes a button or a touch screen for interactions between a user and the device.

In a further technical solution of the present disclosure, core assemblies of the camera module include a processing chip, a control chip, a power supply management chip, and two groups of data encryption chips; the processing chip is in communication connection with the control chip to cooperatively work; the processing chip is matched with an external interface, the WiFi module, and a Serial Peripheral Interface (SPI) flash memory; the control chip is connected to a Complementary Metal-Oxide-Semiconductor Transistor (CMOS) module; the power supply management chip is electrically connected to the processing chip and the control chip; the two groups of data encryption chips respectively serve the processing chip and the control chip; the design enables the processing chip and the control chip to complement advantages; and a chip S3C2410 is used for assisting in processing to reduce a load on a Central Processing Unit (CPU) ASC8850, improve the efficiency of the CPU, achieve system balance processing, make the CPU run faster, and reduce power consumption.

In a further technical solution of the present disclosure, the application uses a built-in eardrum recognition algorithm to recognize and retrieve the image transmitted by a personal protective device, and calculates a probability of finding the eardrum; the algorithm is based on an advanced artificial intelligence technology and a deep learning model of a convolutional neural network; by training a large amount of labeled data, the application has a capability of accurately recognizing the eardrum under various different lighting conditions and angles; after the eardrum is found, the eardrum recognition algorithm makes repeated judgment to filter out mis-recognition; and if filtered data indicates that the ear pick is indeed close to the eardrum, a user is prompted to get away from the eardrum by an icon, a color, a sound, and vibration.

In a further technical solution of the present disclosure, content to be updated, a scope to be updated, and a target device to be updated are determined; a firmware package and related tool required for the update further need to be prepared; a user needs to download the firmware package to a computer or another storage device, then transfer the firmware package to the target device through wireless connection, then initiate an update program on the device, and complete a firmware installation process according to prompts; and after the update is completed, the device needs to be verified and tested to ensure that the firmware update is successful and the device operates normally.

In a further technical solution of the present disclosure, a Bluetooth or WiFi function of the mobile phone or smart device is activated; a wireless signal of the visual ear pick is searched and connected in the APP; the visual ear pick is gently placed at an entrance of the ear canal in a manner of not inserting too deeply to avoid injury of the ear canal or the eardrum; then, the camera in the visual ear pick captures the image inside the ear canal; captured image information is modulated by a signal processing module inside the visual ear pick and is converted into a high-frequency signal suitable for wireless transmission; and a high-frequency signal obtained by modulation is sent out through a wireless transmission module of the visual ear pick to establish a wireless connection with the mobile phone or the smart device.

In a further technical solution of the present disclosure, the application is opened to enter a device connection interface; according to a prompt of the application, a camera of the mobile phone is used to scan a QR code on the device, or an ID number of the device is manually entered to wait for the application to establish a connection with the device; after the connection is successful, the application usually displays information about the connected device; a user views a state and battery level of the device through the application; options for controlling the camera are found in the application; when a button “Camera ON” is tapped, the camera starts to work and captures images; when a button “Camera OFF” is tapped, the camera stops working; an option for adjusting brightness is found in the application; a sliding bar, a button, or another control is used to adjust the brightness of the camera according to an actual need; an adjusted image effect is previewed in real time to ensure clear images and appropriate brightness.

### Beneficial Effects

Through the present disclosure, a user can intuitively observe the condition inside the car canal. The innovative connection method not only improves the efficiency of connection and reduces the steps of user operation, but also significantly enhances the convenience and intuitiveness of the user experience, making it easy for non-skillful users to connect the device to the application. Timely reminders during use can keep a user moving the ear pick away from the eardrum, so that it is safer. The wireless firmware upgrade in the later stage allows a user to enjoy timely function updates.

### BRIEF DESCRIPTION OF THE DRAWINGS

FIG. 1 is a flowchart of an ear pick according to the present disclosure;

FIG. 2 is a schematic structural diagram of a camera module;

FIG. 3 is a schematic diagram of an algorithm encryption implementation model; and

FIG. 4 is a schematic diagram of a communication process of a traditional algorithm.

### DETAILED DESCRIPTION OF THE EMBODIMENTS

The preferred embodiments of the present disclosure will be described below with reference to the accompanying drawings. It should be understood that the embodiments described here are only used to illustrate and explain the present disclosure and are not intended to limit the present disclosure.

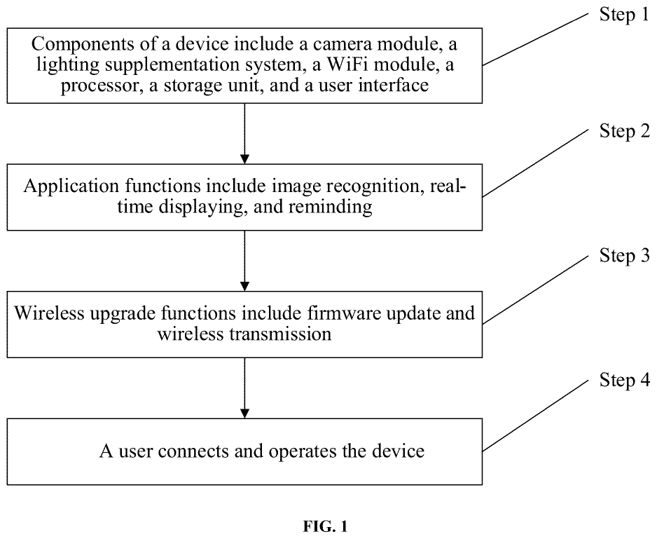

As shown in FIG. 1, a visual ear pick with built-in WiFi, lighting supplementation, and camera functions includes the following steps:
step I, components of the device include a camera module, a lighting supplementation system, a WiFi module, a processor, a storage unit, and a user interface;
step II, application functions include image recognition, real-time displaying, and reminding;
step III, wireless upgrade functions include firmware update and wireless transmission; and
step IV, a user quickly establishes connection to an application through a QR code or by entering a device ID, and the user turns on/turns off a camera to adjust brightness by performing an operation of controlling a device through the application.

Further, a high-resolution camera is arranged in the camera module to capture an image inside the ear canal; the lighting supplementation system includes a light-emitting diode (LED) lamp or another light source to provide sufficient lighting in a low-light environment; the WiFi module is configured to achieve wireless connection between the device and a smart phone or another smart device; the processor is configured to: process image data captured by the camera and control other functions of the device; the storage unit is configured to store firmware and user data; and the user interface includes a button or a touch screen for interactions between a user and the device.

In a specific embodiment, the camera is located at a head portion of the ear pick and can rotate 360 degrees, so that images inside the ear canal can be captured in different angles. The lighting supplementation system automatically adjusts the brightness according to environment lighting to ensure clear images. The WiFi module supports the standard 802.11b/g/n to ensure a stable wireless connection. The processor is responsible for image processing and transmission, as well as managing other functions of the device. The application supports both an iOS platform and an Android platform, and provides an intuitive user interface. The application has an image recognition algorithm that can accurately recognize the eardrum. The application can detect a firmware version of the device and automatically download and update firmware without user intervention. The update process is automatically carried out when the device is connected to a WiFi network. A user can control the function of the ear pick through a simple operation such as sliding and tapping in the application. The application provides historical records and data analysis to help a user track a hygiene state of the ear.

Further, core assemblies of the camera module include a processing chip, a control chip, a power supply management chip, and two groups of data encryption chips; the processing chip is in communication connection with the control chip to cooperatively work; the processing chip is matched with an external interface, the WiFi module, and an SPI flash memory; the control chip is connected to a CMOS module; the power supply management chip is electrically connected to the processing chip and the control chip; the two groups of data encryption chips respectively serve the processing chip and the control chip; the design enables the processing chip and the control chip to complement advantages; and a chip S3C2410 is used for assisting in processing to reduce a load on a CPU ASC8850, improve the efficiency of the CPU, achieve system balance processing, make the CPU run faster, and reduce power consumption.

In a specific embodiment, the camera module includes light receiving, digital diagnosis, electrical interface, power supply, light emitting, and wavelength division multiplexing optical assemblies. The light receiving unit is composed of a photoelectric conversion chip, a transimpedance and limiting amplification circuit, and a clock data recovery circuit. The light emitting unit includes a laser emitter, a driving circuit, and a transmission de-emphasis/pre-emphasis and clock data recovery circuit. This module is compatible with SFP+standards at rates of 5G and 24.33G and is suitable for 25 Gb/s Ethernets and next-generation Common Public Radio Interface (CPRI) systems, thereby improving the applicability of the module. In addition, the laser power is directly monitored through circuit design, which replaces a traditional method for using a backlight diode, simplifies the production process, and reduces the cost.

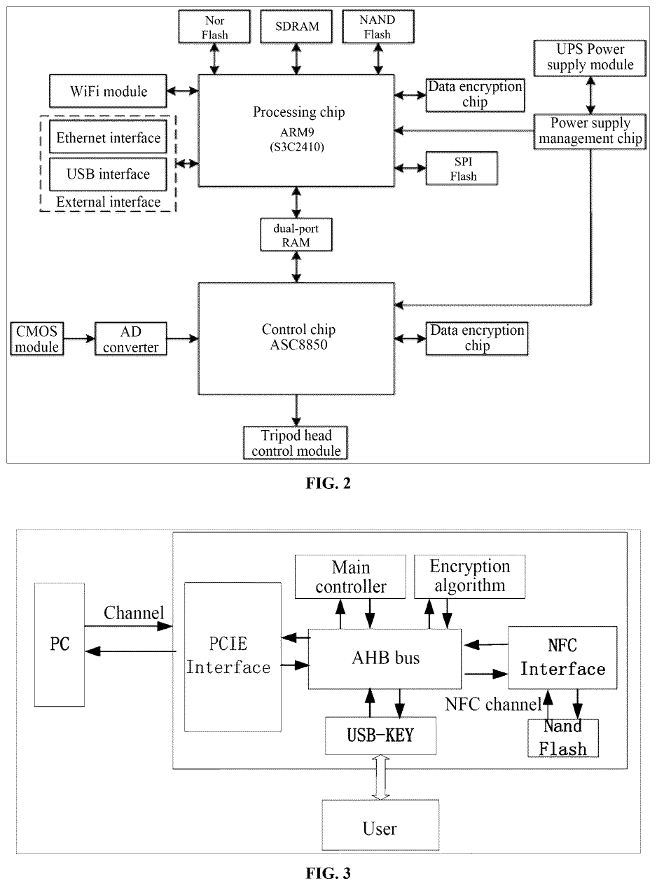

An encryption algorithm is added in a data encoding module. In the field of public key cryptography, Elliptic Curve Cryptography (ECC) provides the highest bit strength. Compared with unit encryption, this algorithm can achieve the same level of encryption using a key that is simpler than a general encryption technology. The ECC is more suitable for key distribution. During encryption, an ECC encryption algorithm can be used to enhance the safety certification strength of the entire system. The implementation model is shown in FIG. 3.

In FIG. 3 of encryption, this study adopts an ECC cryptographic technology system. A main principle is to use a method for positioning by using network data feature attributes to shorten data extraction time of a frame number and expand the data recognition efficiency. A multi-level and multi-objective method is used to increase the randomness and diversity of sharing of power grid procurement data.

An elliptic equation of a finite field is defined in region Y as follows:

$${y}^{2} + {a}_{1} xy + {a}_{3} y = {x}^{3} + {a}_{2} {x}^{2} + {a}_{4} x + {a}_{6} \qquad ( 1 )$$

In formula (1), α₁ to α₆ belongs to Y. In Y, all points satisfy (x, y) in the formula. In a prime field Y=Fₚ, Δ≠0 of the ellipse can simplify the above formula, and a calculation formula is as follows:

$${y}^{2} = {x}^{2} + ax + b \qquad ( 2 )$$

In formula (2), α, b∈Y, Δ=4a³+27b². All solutions satisfy (x, y)∈Fₚ×Fₚ. A center point, i.e. an origin O, of the same ellipse forms a point set of the ellipse on Fₚ, which is represented by Eₚ(α, b). A congruence operation and a logarithmic operation of a primitive root is to obtain an integer k by determining any two points P and Q on Y, making it difficult to form the following expressions by the above parameters. This is also the characteristic of the encryption technology of the algorithm.

$$kP = P + P + L + P = Q \qquad ( 3 )$$

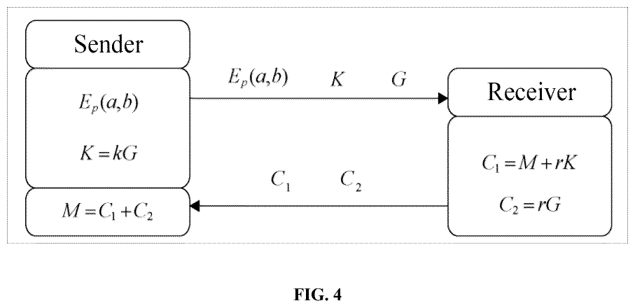

In formula (3), L is a constant. According to the IEEEP1363 ECC standard, an ellipse is selected, and a point on the elliptic line Eₚ(α, b) is selected as a base point G. An integer is randomly selected between [1, n−1] and is used as a private key. K=kG is a public key. A specific communication process is as shown in FIG. 4.

In FIG. 4, the communication module only has parameters such as Eₚ(α, b), k, G, and C. K and G are used to solve k or C2, and G is used to solve r. These will involve solving the congruence operation and a logarithmic operation of the primitive root. Therefore, even if an attacker steals a channel parameter, it is difficult to obtain the real transmission information. This is a security feature of this algorithm. However, a transmission method still needs to be optimized. Improving the transmission rate can provide a better speed assurance for data encryption.

### Improvement on the ECC Encryption Algorithm:

Due to high time complexity of the algorithm, it is not conducive to computation of an embedded electronic device in communication and may increase a communication latency. Therefore, this study optimizes the algorithm. Optimization steps are as follows. The design of a calculation method for a size of a scalar multiplication window in ECC greatly reduces the complexity. A pre-calculation method for a cardinality chain length is applied to scalar multiplication calculation. Calculating an optimal multi-cardinality chain improves the encryption speed. Scalar multiplication rK and rG of the integers r, K, and G are calculated. NAF encoding is performed on integer r. A calculation process is as follows:

$${r}^{j} = {\sum }_{i = 0}^{j} {s}_{i} {2}^{i} , {s}_{i} ∈ ( - 1 , 0 , 1 ) \qquad ( 4 )$$

In formula (4), r^{j} is an encoded integer; r, j are decoded integers; and a bit expression of integer r is i∈[0, j].

The value of window ω is estimated, and a calculation formula is:

$$ω = \frac{log r}{INT ( {E}_{b} )} \qquad ( 5 )$$

In formula (5),

$${E}_{b} = \frac{2 + 3 + 7}{3} ,$$

and r is an integer of the previous process. INT(E_{b}) is to round off a target.

r^{j} is divided into windows according to ω described above, and is a combination of

$${R}_{1}^{j} .$$

An implementation is as follows:

$${r}^{j} = {2}^{d - 1} {R}_{d - 1}^{j} + {2}^{d - 2} {R}_{d - 2}^{j} + … + {2}^{1} {R}_{1}^{j} + {2}^{0} {R}_{0}^{j} \qquad ( 6 )$$

$${R}_{I}^{j} = {2}^{ω - 1} {r}_{I}^{ω - 1} + {2}^{ω - 2} {r}_{I}^{ω - 2} + … + {2}^{1} {r}_{I}^{1} + {2}^{0} {r}_{I}^{0} \qquad ( 7 )$$

In formula (6), r^{j} is an NAF encoded integer r; j is a bit expression length of the decoded integer r,

$$d = \frac{j}{ω} ; {R}_{I}^{j}$$

is an Ith window of r^{j}; and

$${r}_{I}^{ω - 1}$$

is a value ω−1 of the Ith window.

$${R}_{I}^{j}$$

with a base of [2,3,7] is set to calculate a cardinality chain of a window. First, an existence characteristic of xP is evaluated in Table T pre-constructed by the system. The system starts to operate in a case that xP exists. Otherwise, the cardinalities and the value of point P of all sets x∈[1, 2, 3, 7, . . . , 2^{bi}3^{ci}7^{di}] are recorded in Table T. Then, a length of a target chain is obtained by pre-conversion.

An expression of the longest chain of the cardinality chain after pre-conversion is:

s=\sqrt{log Rₗ^{j}/log log Rₗ^{j}} (8)

In formula (8), s is the longest chain.

The cardinality chain is calculated using a greedy algorithm. An optimal multi-cardinality chain of window

$${R}_{I}^{j}$$

is calculated according to a relevant mathematic model:

$$\{ \begin{matrix}{R}_{1}^{j} = {\sum }_{i = 1}^{n} {s}_{i} {2}^{bi} {3}^{ci} {7}^{di} \\ {C}_{i} = max \frac{2 {N}_{b} + 3 {N}_{c} + 7 {N}_{d}}{2 + 3 + 7}\end{matrix} \qquad ( 9 )$$

In formula (9), sᵢ is a value between −1 and 1; n is a length of a real cardinality chain, between 1 and s. i∈[1, n], Cᵢ is an optimal evaluation of the cardinality chain; N_{b} N_{c}, and N_{d} are number features of 0 in sets bi, ci, and di.

rK and rG are calculated through the optimal cardinality chain of Rₗ^{j}.

Calculation formulas are as follows:

$$rK = {\sum }_{I = 1}^{d} {R}_{1}^{j} K \qquad ( 10 )$$

$$rG = {\sum }_{I = 1}^{d} {R}_{1}^{j} G \qquad ( 11 )$$

In formulas (10) and (11), G is any point on the ellipse, and K is a system public key. Finally, secure decryption of data is achieved.

Based on any variable obtained by homomorphic encryption, shared dynamic multi-level features of the model are set. A multi-objective and multi-level sharing strategy is constructed. An encryption target of the power grid procurement data is used as a direction. A data sharing link is set in the model. A plurality of data source nodes are fused to further optimize a data sharing feature using an HBT technology. However, this technology may have a key distribution issue. A key is easily copied and is not convenient for security management.

Further, the application uses a built-in eardrum recognition algorithm to recognize and retrieve the image transmitted by a personal protective device, and calculates a probability of finding the eardrum; the algorithm is based on an advanced artificial intelligence technology and a deep learning model of a convolutional neural network; by training a large amount of labeled data, the application has a capability of accurately recognizing the eardrum under various different lighting conditions and angles; after the eardrum is found, the eardrum recognition algorithm makes repeated judgment to filter out mis-recognition; and if filtered data indicates that the ear pick is indeed close to the eardrum, a user is prompted to get away from the eardrum by an icon, a color, a sound, and vibration.

In a specific embodiment, model training is a key step in the eardrum recognition algorithm. A specific process includes:
i. Data acquisition: acquiring various ear canal images under different lighting conditions, angles, backgrounds, and ear canal structures. These images are acquired through the device during actual use and are labeled by a professional to clearly indicate the position of the eardrum.
ii. Data preprocessing: preprocessing the acquired images, including normalization and data augmentation to increase the diversity and robustness of the data.
iii. Model construction: constructing an image classification model using a convolutional neural network. The structure of the model includes several convolutional layers, a pooling layer, and a fully connected layer for extracting image features and performing classification.
iv. Model training: inputting the preprocessed data into the model for training, and continuously adjusting model parameters through a backpropagation algorithm to accurately recognize the eardrum. In the training process, a cross validation method is used to evaluate the performance of the model and adjust hyperparameters to optimize the effect of the model.
v. Model validation and testing: evaluating the model using an independent validation set and test set to ensure high accuracy even on unseen data. The model is comprehensively evaluated through indexes such as a confusion matrix, an accuracy, a recall rate, and an F1 score.

Multi-frame validation is an important step in eardrum recognition. The algorithm continuously detects the eardrum in multiple frames of images to avoid single-frame misjudgment and improve the recognition accuracy. Specific steps include:
i. Continuous detection: detecting the eardrum from each frame of image within a time window, and recording detection results of each frame.
ii. Result integration: performing statistical analysis on the detection results of all the frames to determine whether the detection results satisfy set threshold conditions. For example, the eardrum has been detected in at least three out of five consecutive frames.
iii. Final determination: confirming, according to a statistical result, presence or absence of the eardrum to avoid an error prompt caused by single-frame misjudgment.

The algorithm design for filtering and parameter settings are the core of multi-frame filtering. A filter determines the presence of the eardrum by setting a time window and a threshold. Specific steps include:
i. Time window selection: setting an appropriate time window length according to system performance and user experience.
ii. Threshold setting: setting a proper threshold to ensure the stability and reliability of the detection results within the time window.
iii. Selection of a filtering algorithm: Kalman filtering or weighted average filtering is used to smooth the detection results of several previous frames and the detection result of the current frame, thereby improving the stability and accuracy of a recognition result.

After the eardrum has been detected, the application may remind a user to move the ear pick away from the eardrum through an icon, a color change, a sound, or vibration, to ensure safe operations. A specific process includes:
i. Icon prompt: A prominent warning icon is displayed on an application interface. When the eardrum has been detected, the icon will flash or change color to attract the attention of a user.
ii. Color change: A background color of the interface or colors of some key regions will change, such as from green to red, indicating that a user needs to stop the operation and adjust the position of the ear pick.
iii. Sound alarm: The system will issue a sound alarm, such as a beep or voice prompt, to remind a user of operation safety.
iv. Vibration reminder: If the device supports a vibration function, the system will trigger a vibration reminder when the eardrum has been detected, so that a user feels warning information.

Further, content to be updated, a scope to be updated, and a target device to be updated are determined; a firmware package and related tool required for the update further need to be prepared; a user needs to download the firmware package to a computer or another storage device, then transfer the firmware package to the target device through wireless connection, then initiate an update program on the device, and complete a firmware installation process according to prompts; and after the update is completed, the device needs to be verified and tested to ensure that the firmware update is successful and the device operates normally.

In a specific embodiment, the application can download new firmware from a server and automatically update the device. After the personal protective device is powered on and connected to the application, the application may read the model and version number of the device. Then, the application requests the version number of the firmware of the device to be remodeled from the server. If new firmware with a new version exists, a user will be prompted to perform a wireless firmware upgrade. Once the user agrees with the upgrade, the application running on the mobile phone or the tablet will download the latest firmware from the server and notify the personal protective device to enter a wireless upgrade mode through Bluetooth or WiFi. After the personal protective device makes a response, the application starts to send the firmware for upgrade. After receiving the new firmware, the personal protective device performs data verification. After confirmation, the firmware is written into an internal storage chip to complete the firmware update.

Further, a Bluetooth or WiFi function of the mobile phone or smart device is activated; a wireless signal of the visual ear pick is searched and connected in the APP; the visual ear pick is gently placed at an entrance of the ear canal in a manner of not inserting too deeply to avoid injury of the ear canal or the eardrum; then, the camera in the visual ear pick captures the image inside the ear canal; captured image information is modulated by a signal processing module inside the visual ear pick and is converted into a high-frequency signal suitable for wireless transmission; and a high-frequency signal obtained by modulation is sent out through a wireless transmission module of the visual ear pick to establish a wireless connection with the mobile phone or the smart device.

In a specific embodiment, wireless image transmission from an internal camera of the personal protective device to the mobile phone or the tablet includes the following steps:
1. Waterproof miniature camera module with supplement lamps. The camera module is mounted inside a sleeve at a tip end of the personal protective device. The sleeve can be made of plastic or metal. An optical part of the camera module is surrounded by a circle of supplement lamps. The supplement lamps can adjust the brightness according to an actual need. The camera and the supplement lamps are covered with transparent glue to achieve a waterproof effect. Lighting has a sterilizing function. During use, it can effectively reduce the presence of bacteria and microorganisms, ensuring the safety and hygiene of use. The tip end of the ear pick can be provided with a conventional ear pick and a micro tweezer, which can effectively pick up a foreign object in the ear canal. The tweezer is made of high-strength stainless steel, which ensures that it will not bend or be broken during use. A user can manually control the tweezer to be opened and closed during operation. This greatly improves the versatility and practicality of the ear pick.
2. After the main control chip is powered on, the camera module is initialized through an Inter-Integrated Circuit (IIC) bus. Working parameters of the camera are set, such as a color, a frame rate, brightness, and an image size. After the settings are completed, the camera is activated for image acquisition through an IIC command. The supplementation lamps are turned on simultaneously, and the brightness is adjusted and set on the APP according to a user need. After receiving a brightness command, the main control chip adjusts the brightness of the supplementation lamps through Pulse Width Modulation (PWM). The camera module transmits raw image data to the main control chip through an interface such as SPI/DVP/MIPI. The volume of the original image dynamically changes as details and colors of a captured target change The original image occupies a too large space and does not use data transmission. Therefore, a built-in image encoder of the main control chip is used to compress the original image and reduce the volume. The method for compressing an image is JPEG/H264/H265, and the volume of the compressed image is reduced to a fraction to one-tenth of the original volume, and then the image is transmitted, through WiFi transmission, to the APP of the mobile phone for displaying.
3. The main chip establishes a data transmission channel by connecting a WiFi hotspot to the device such as the mobile phone and the tablet that can run the application.
4. The application can observe video data transmitted by the personal protective device in real time and perform operations such as mirroring, zooming, photo taking, and video recording.

Further, the application is opened to enter a device connection interface; according to a prompt of the application, a camera of the mobile phone is used to scan a QR code on the device, or an ID number of the device is manually entered to wait for the application to establish a connection with the device; after the connection is successful, the application usually displays information about the connected device; a user views a state and battery level of the device through the application; options for controlling the camera are found in the application; when a button “Camera ON” is tapped, the camera starts to work and captures images; when a button “Camera OFF” is tapped, the camera stops working; an option for adjusting brightness is found in the application; a sliding bar, a button, or another control is used to adjust the brightness of the camera according to an actual need; an adjusted image effect is previewed in real time to ensure clear images and appropriate brightness.

In a specific embodiment, the design of the user interface is a main presentation method of a user operation process, which specifically includes the following aspects:
i. Interface layout: A concise and clear interface layout is designed. A warning icon is placed in a prominent position such as a center or an upper right corner of a screen.
ii. Prompt information: A brief text prompt is displayed next to the warning icon, such as “Near the eardrum. Please adjust the position”, to ensure that a user can quickly understand the warning information.
iii. Visual effect: A vivid color change and an animation effect are used to enhance the warning effect, so that a user can notice the warning prompt in a timely manner during the operation.
iv. Multimodal reminder: It ensures that a user can receive effective warning information in various operation environments in various reminding manners such as an icon, a color, a sound, and vibration.

What is claimed is:

1. A visual ear pick with built-in WiFi, lighting supplementation, and camera functions, comprising the following key components and steps:
step I, components of a device comprise a camera module, a lighting supplementation system, a WiFi module, a processor, a storage unit, and a user interface;
step II, application functions comprise image recognition, real-time displaying, and reminding;
step III, wireless upgrade functions comprise firmware update and wireless transmission; and
step IV, a user quickly establishes connection to an application through a QR code or by entering a device ID, and the user turns on/turns off a camera to adjust brightness by performing an operation of controlling the device through the application.

2. The visual ear pick with the built-in WiFi, lighting supplementation, and camera functions according to claim 1, wherein
in step I, a high-resolution camera is arranged in the camera module to capture an image inside an ear canal; the lighting supplementation system comprises a light-emitting diode (LED) lamp or another light source to provide sufficient lighting in a low-light environment; the WiFi module is configured to achieve wireless connection between the device and a smart phone or another smart device; the processor is configured to: process image data captured by the camera and control other functions of the device; the storage unit is configured to store firmware and user data; and the user interface comprises a button or a touch screen for interactions between the user and the device.

3. The visual ear pick with the built-in WiFi, lighting supplementation, and camera functions according to claim 2, wherein
core assemblies of the camera module comprise a processing chip, a control chip, a power supply management chip, and two groups of data encryption chips; the processing chip is in communication connection with the control chip to cooperatively work; the processing chip is matched with an external interface, the WiFi module, and a Serial Peripheral Interface (SPI) flash memory; the control chip is connected to a Complementary Metal-Oxide-Semiconductor Transistor (CMOS) module; the power supply management chip is electrically connected to the processing chip and the control chip; the two groups of data encryption chips respectively serve the processing chip and the control chip; the design enables the processing chip and the control chip to complement advantages; and a chip S3C2410 is used for assisting in processing to reduce a load on a Central Processing Unit (CPU) ASC8850, improve an efficiency of the CPU, achieve system balance processing, make the CPU run faster, and reduce power consumption.

4. The visual ear pick with the built-in WiFi, lighting supplementation, and camera functions according to claim 1, wherein
in step II, the application uses a built-in eardrum recognition algorithm to recognize and retrieve an image transmitted by a personal protective device, and calculates a probability of finding an eardrum; the built-in eardrum recognition algorithm is based on an advanced artificial intelligence technology and a deep learning model of a convolutional neural network; by training a large amount of labeled data, the application has a capability of accurately recognizing the eardrum under various different lighting conditions and angles; after the eardrum is found, the built-in eardrum recognition algorithm makes repeated judgment to filter out mis-recognition; and when filtered data indicates that the visual ear pick is indeed close to the eardrum, the user is prompted to get away from the eardrum by an icon, a color, a sound, and vibration.

5. The visual ear pick with the built-in WiFi, lighting supplementation, and camera functions according to claim 1, wherein
in step III, content to be updated, a scope to be updated, and a target device to be updated are determined; a firmware package and related tool required for the update further need to be prepared; the user needs to download the firmware package to a computer or another storage device, then transfer the firmware package to the target device through wireless connection, then initiate an update program on the device, and complete a firmware installation process according to prompts; and after the update is completed, the device needs to be verified and tested to ensure that the firmware update is successful and the device operates normally.

6. The visual ear pick with the built-in WiFi, lighting supplementation, and camera functions according to claim 1, wherein
in step III, a Bluetooth or WiFi function of a mobile phone or a smart device is activated; a wireless signal of the visual ear pick is searched and connected in the APP; the visual ear pick is gently placed at an entrance of an ear canal in a manner of not inserting too deeply to avoid injury of the ear canal or an eardrum; then, the camera in the visual ear pick captures an image inside the ear canal; captured image information is modulated by a signal processing module inside the visual ear pick and is converted into a high-frequency signal suitable for wireless transmission; and the high-frequency signal obtained by modulation is sent out through a wireless transmission module of the visual ear pick to establish a wireless connection with the mobile phone or the smart device.

7. The visual ear pick with the built-in WiFi, lighting supplementation, and camera functions according to claim 4, wherein
in step IV, the application is opened to enter a device connection interface; according to a prompt of the application, a camera of a mobile phone is used to scan the QR code on the device, or an ID number of the device is manually entered to wait for the application to establish a connection with the device; after the connection is successful, the application usually displays information about the device; the user views a state and battery level of the device through the application; options for controlling the camera are found in the application; when a button “Camera ON” is tapped, the camera starts to work and captures images; when a button “Camera OFF” is tapped, the camera stops working; an option for adjusting brightness is found in the application; a sliding bar, a button, or another control is used to adjust the brightness of the camera according to an actual need; an adjusted image effect is previewed in real time to ensure clear images and appropriate brightness.
